(19) United States
(12) Patent Application Publication
(10) Pub. No.: US 2024/0101447 A1
(43) Pub. Date: Mar. 28, 2024

(54) NOZZLE

(71) Applicant: Crystal IS, Inc., Green Island, NY (US)

(72) Inventors: James Davis, Green Island, NY (US); Rajul V. Randive, Niskayuna, NY (US); Richard M. Mariita, Troy, NY (US)

(21) Appl. No.: 18/371,954

(22) Filed: Sep. 22, 2023

Related U.S. Application Data
(60) Provisional application No. 63/409,624, filed on Sep. 23, 2022.

(51) Int. Cl. C02F 1/32; A61L 2/10; A61L 2/26

(52) U.S. Cl. CPC C02F 1/325; A61L 2/10; A61L 2/26; A61L 2202/11; C02F 2201/3222; C02F 2201/3228; C02F 2307/10

(57) ABSTRACT

A liquid treatment system has a nozzle body comprised of a semi-translucent material. The nozzle body has a side wall and a tip forming an inner chamber and an outer surface. The side wall thickness and tip thickness are a function of an anti-microbial light intensity range of the outer surface of the nozzle body. The liquid treatment system also has a light circuit coupled to the side wall. The light circuit emits an anti-microbial light toward the inner chamber.

### PRIORITY

This patent application claims priority from provisional U.S. patent application No. 63/409,624, filed Sep. 23, 2022, entitled, “NOZZLE,” and naming James Davis et al. as inventors, the disclosure of which is incorporated herein, in its entirety, by reference.

### FIELD

Illustrative embodiments of the invention generally relate to liquid treatment and, more particularly, various embodiments of the invention relate to using ultraviolet light to prevent retrograde contamination.

### BACKGROUND

Fluids, including liquid water, are commonly used for many domestic, commercial, and industrial purposes, such as drinking, food preparation, manufacturing, processing of chemicals, and cleansing. It is often necessary to purify a liquid prior to its use. Filters such as ceramic filters are typically used to remove particulate and chemical impurities from liquids. In addition, a liquid can be exposed to UV radiation to neutralize microbial growth (microorganisms and deleterious pathogens that may be present in the liquid, e.g., bacteria, viruses, and protozoa). Exposure to certain wavelengths of light can disrupt the DNA of many cellular microorganisms—virtually destroying them or rendering them substantially harmless. The exposure to UV radiation can also substantially prohibit the growth and/or reproduction of microorganisms in the liquid. Treatment of the liquid alone may be insufficient to dispense purified water. For example, contamination of the nozzle from which the liquid is dispensed may reintroduce microbial growth into the liquid as it is dispensed.

### SUMMARY OF VARIOUS EMBODIMENTS

In accordance with one embodiment of the invention, a liquid treatment system has a nozzle body comprised of a semi-translucent material. The nozzle body has a side wall and a tip forming an inner chamber and an outer surface. The side wall thickness and tip thickness are a function of an anti-microbial light intensity range of the outer surface of the nozzle body. The liquid treatment system also has a light circuit coupled to the side wall. The light circuit emits an anti-microbial light toward the inner chamber.

The semi-translucent material may have a reflectivity in a range of 80-98%.

In some embodiments, the light circuit includes a light emitting diode configured to emit anti-microbial light including ultraviolet-C light. The anti-microbial light which has passed through the nozzle body to the outer surface of the nozzle body may have an intensity within the anti-microbial light intensity range. The anti-microbial light may disinfect the outer surface of the nozzle body while not exceeding 3 mJ/cm² at the outer surface over an 8-hour period.

In some embodiments, the tip includes a plurality of apertures.

The light circuit may be at least partially inserted into the side wall. In some embodiments, the side wall has a thinned section and the light circuit emits the anti-microbial light toward the inner chamber through the thinned section of the side wall. This light circuit may have an LED and a spacer, where the spacer is positioned between the thinned section of the side wall and the LED.

In some embodiments, the side wall includes an aperture, and the light circuit is inserted into the aperture. This light circuit may have a translucent window and a housing coupled to the translucent window.

In accordance with another embodiment of the invention, a method for operating a nozzle couples a light circuit to a nozzle body including a side wall and a tip. The side wall and the tip forming an outer surface and an inner chamber. The method emits an anti-microbial light toward the inner chamber of the nozzle body. The method transmits a portion of the anti-microbial light to the outer surface of the nozzle body through the nozzle body. The method disinfects the outer surface of the nozzle body after transmitting the portion of the anti-microbial light. The portion of the anti-microbial light has an intensity less than 3 mJ/cm² over an 8-hour period.

The portion of the anti-microbial light may be a function of a nozzle body material, a tip thickness, and a side wall thickness. The nozzle body material may consist of a semi-translucent material with a reflectivity in a range of 80-98%.

Emitting the anti-microbial light may occur after determining a standby time period.

In some embodiments, the light circuit includes a light emitting diode (LED) to emit ultraviolet-C light. In some embodiments, the light circuit includes an LED and a spacer and coupling the light circuit to the nozzle body includes positioning the spacer between the thinned section of the side wall and the LED.

In some embodiments, the tip includes a plurality of apertures.

Coupling the light circuit to the nozzle body may include at least partially inserting the light circuit into the side wall. In some embodiments, the side wall has a thinned section, and emitting the anti-microbial light toward the inner chamber of the nozzle body includes emitting the anti-microbial light through the thinned section of the side wall.

In some embodiments, the side wall includes an aperture, and coupling the light circuit to the nozzle body includes inserting the light circuit into the aperture.

### BRIEF DESCRIPTION OF THE DRAWINGS

Those skilled in the art should more fully appreciate advantages of various embodiments of the invention from the following “Description of Illustrative Embodiments,” discussed with reference to the drawings summarized immediately below.

FIG. 1 schematically shows a liquid treatment system in accordance with various embodiments.

FIG. 2 is a cross-sectional view schematically showing a nozzle with a thinned section in accordance with various embodiments.

FIG. 3 is an exploded view schematically showing the nozzle with the thinned section in accordance with various embodiments.

FIG. 4 is a cross-sectional view schematically showing a nozzle with a side wall aperture in accordance with various embodiments.

FIG. 5 is an exploded view schematically showing the nozzle with the side wall aperture in accordance with various embodiments.

FIG. 6 is a side view schematically showing the nozzle with the side wall aperture in accordance with various embodiments.

FIG. 7 is a flowchart showing a process for operating a nozzle in accordance with various embodiments.

### DESCRIPTION OF ILLUSTRATIVE EMBODIMENTS

In illustrative embodiments, a nozzle for a liquid treatment system disinfects the outside and inside of a nozzle body using anti-microbial light, such as ultraviolet-C (UVC) light, in order to prevent or eliminate retrograde contamination from microbial growth. A light emits the anti-microbial light into the nozzle body. The nozzle body is comprised of a semi-translucent material that allows a safe level of anti-microbial light to pass through the nozzle body to the outer surface of the nozzle body, including the outer surface of the tip. The anti-microbial light is sufficient to disinfect or prevent contamination from microorganisms originating outside of the liquid treatment system from growing on the nozzle and forming biofilm on the inside of the nozzle body as well as the outer surface of the nozzle body. By preventing the growth of biofilm on the outside of the nozzle body, the need for users to touch the nozzle body while cleaning or spraying aerosols cleaners that would introduce chemicals to the nozzle is reduced. Details of illustrative embodiments are discussed below.

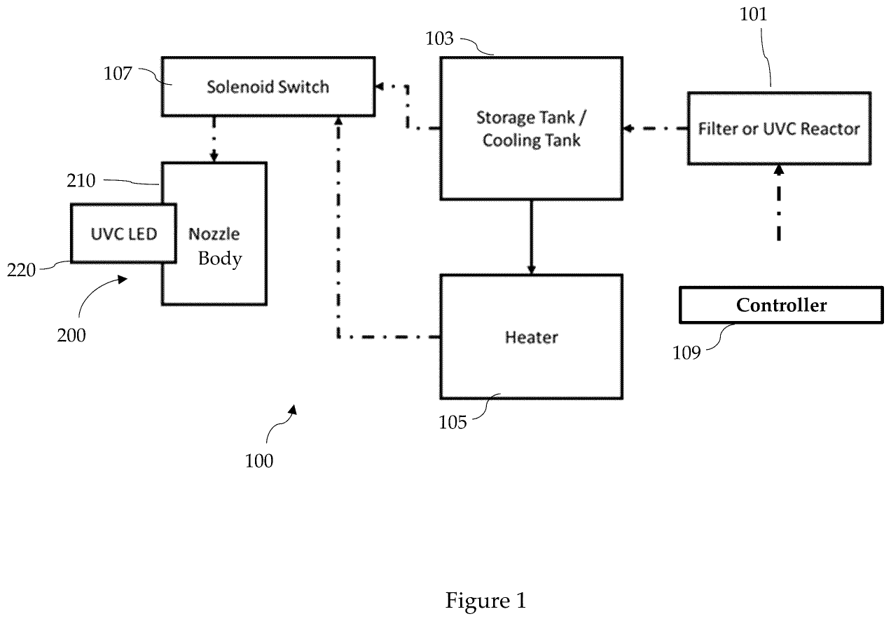

FIG. 1 schematically shows a liquid treatment system 100 configured to purify a liquid and provide a liquid on demand in accordance with various embodiments. The liquid, among other things, may be water. It should be appreciated the liquid treatment system 100 may have more or fewer components, or may be arranged in a different configuration.

The liquid treatment system 100 has an initial filter stage 101 to remove particles or chemicals from fluid received by the liquid treatment system 100. The liquid treatment system 100 has a holding tank stage 103 configured to store fluid in a tank, and may be configured to cool the stored liquid. The liquid treatment system 100 also has a heater stage 105 configured to heat the liquid flowing through the heater stage 105. The liquid treatment system 100 further includes a solenoid switch stage 107 configured to selectively allow the flow of heated fluid from the heater stage 105 or liquid from holding tank stage 103.

The liquid treatment system 100 also includes a nozzle 200 configured to dispense the liquid flowing through the solenoid switch stage 107. The nozzle 200 has a nozzle body 210 configured to form a flow path for the liquid being dispensed from the liquid treatment system 100. The nozzle 200 also has a light circuit 220 configured to dose the nozzle body 210 and the liquid with anti-microbial light configured to disinfect the nozzle 200 and the fluid.

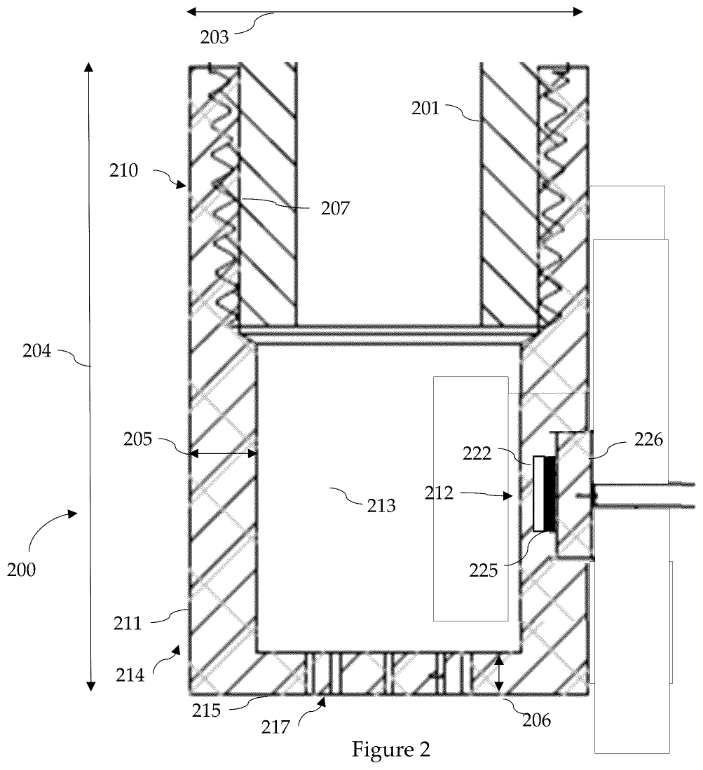

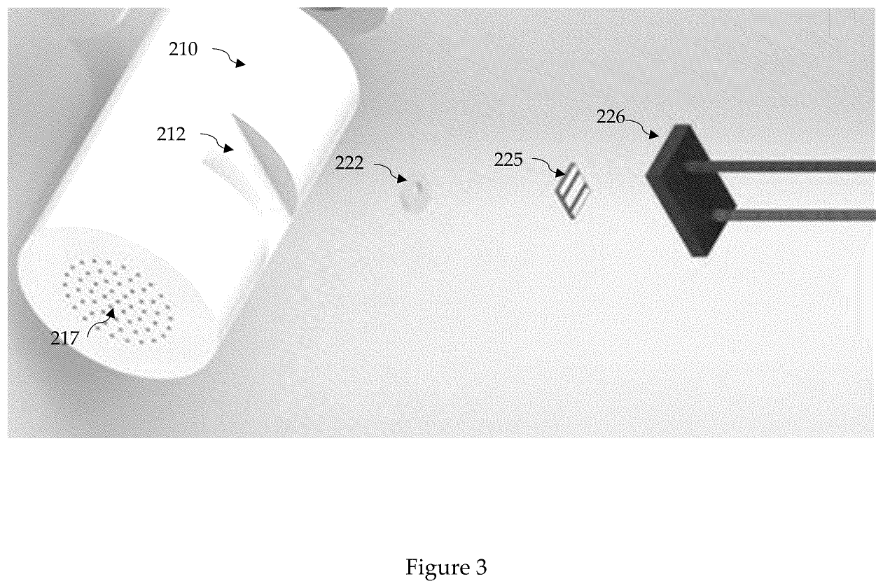

FIGS. 2 and 3 schematically show the nozzle 200 in accordance with various embodiments. The nozzle 200 has a height 204, a width 203, and thicknesses 205, 206. It should be appreciated that the nozzle 200 may include different component characteristics or include more or fewer components than the components illustrated in FIGS. 2 and 3. In other embodiments, the nozzle 200 may have a different thickness, a different number of tip apertures, a different height, a different width, or a different degree to which the light circuit 220 is inserted into a thinned section 212, among other things.

The nozzle 200 is coupled to a connection point 201 of the liquid treatment system 100 by way of a connection surface 207 of the nozzle 200. In the illustrated embodiment, the connection surface 207 is a threaded surface. In other embodiments, the nozzle 200 may be connected to the remainder of the liquid treatment system 100 by another means.

The nozzle body 210 includes a side wall 211 and a tip 215 forming an inner chamber 213 and an outer surface 214. The tip 215 includes tip apertures 217 allowing the flow of liquid from the nozzle 200.

The nozzle body 210 may be comprised of a material that is partially translucent and partially reflective to allow a reduced intensity of light to transmit to an outer surface 214 of the nozzle body 210. Among other things, the material may be polytetrafluoroethylene. The material of the nozzle body 210 may be a type of material having a reflectance to transmission ratio near 12:1. In some embodiments, the nozzle body 210 includes a material having a reflectivity between 80-98%, among other things. In some embodiments, the nozzle body 210 solely consists of the semi-translucent material. In some embodiments, the outer surface 214 consists of the same semi-translucent material as other portions of the nozzle body 210. In some embodiments, the outer surface 214 does not include a coating to prevent retrograde contamination.

In order to mitigate the health risks of exposure to anti-microbial light, the intensity of anti-microbial light used to dose the outer surface 214 of the nozzle body 210 must be limited to a safe threshold. At the same time, the anti-microbial light must have an intensity sufficient to sanitize the nozzle body 210 including the outer surface 214. For these reasons, nozzle 200 may be configured such that the intensity of the anti-microbial light reaching the outer surface 214 of the nozzle body 210 is within a range inclusive of 0.9-3.0 mJ/cm².

The sidewall thickness 205 and tip thickness 206 are configured to allow a safe intensity of anti-microbial light to dose the outer surface 214. In some embodiments, the side wall thickness 205 is equal to the tip thickness 206. In some embodiments, the thicknesses of the side wall 211 and tip 215 are uniform. The nozzle body thicknesses 205, 206 are a function of the material comprising the nozzle body 210, the intensity of light inside the nozzle body 210, and the desired intensity of light dosing the outside surface of the nozzle body 210. In some embodiments, the nozzle body thicknesses 205, 206 are within a range inclusive of 2 mm and 5 mm. In some embodiments, the nozzle body 210 may have a varying thickness that may be determined as a function of light intensity requirements. In embodiments where the internal illumination is nearly uniform from highly reflective and diffuse inner surfaces, beer's law below may be used to determine the nozzle body thicknesses 205, 206:

T=10^{∈l} (1)

Such that T is the transmission of the side wall, is the attenuation coefficient for the side wall material, and L is the thickness of the side wall 211 or tip 215. The transmission should be such that the transmission through the side wall 211 is equal or nearly equal to the transmission through the tip apertures 217.

The light circuit 220 is configured to be inserted into a cavity in the nozzle body 210 formed by the thinned section 212 of the side wall 211, the cavity being sized to receive the light circuit 220. The thinned section 212 is a portion of the side wall 211 having a reduced thickness compared to the remainder of the side wall 211. By emitting light through the thinned section 212 instead of another portion of the side wall 211, the inner chamber 213 receives sufficient anti-microbial light intensity to disinfect the liquid and the inner chamber 213.

In the illustrated embodiment, the thinned section 212 includes a rectangular cross-section, but in other embodiments, the cross-section of the thinned section 212 may be a different shape, thickness, or size, among other things. The thinned section 212 may have a thickness to maintain structural integrity of the nozzle body 210 while maximizing the intensity of the anti-microbial light emitted into the inner chamber 213 by the lamp 225.

The light circuit 220 includes a lamp 225 configured to emit anti-microbial light. In the illustrated embodiment, the lamp 225 includes a light emitting diode (LED). In some embodiment, the LED is configured to emit ultraviolet light, such ultraviolet light in the UVC frequency range. In some embodiments, the lamp 225 may include other sources of light configured to emit anti-microbial light, such as a mercury-vapor lamp, among other things. The lamp 225 is coupled to a printed circuit board (PCB) 226 configured to operate the lamp 225. For example, the PCB 226 may provide power to the lamp 225. The lamp 225 may consume power at an average rate within a range inclusive of 0.35-3.5 watts, to name but one example. The lamp 225 may also emit UVC light having an optical power within a range inclusive of 5-20 mW, to name but one example.

The light circuit 220 may include a spacer 222 positioned between the side wall 211 and the lamp 225. The spacer 222 may absorb and disperse heat generated by the lamp 225 or PCB 226. The spacer 222 may also aid the coupling between the lamp 225 and the thinned section 212.

The lamp 225 may be coupled to the spacer 222 by an adhesive, to name but one example. The spacer 222 may be coupled to the side wall 211 by an adhesive, to name but one example. In some embodiments, the light circuit 220 may be coupled to the thinned section 212 by press fitting the light circuit 220 into the thinned section 212.

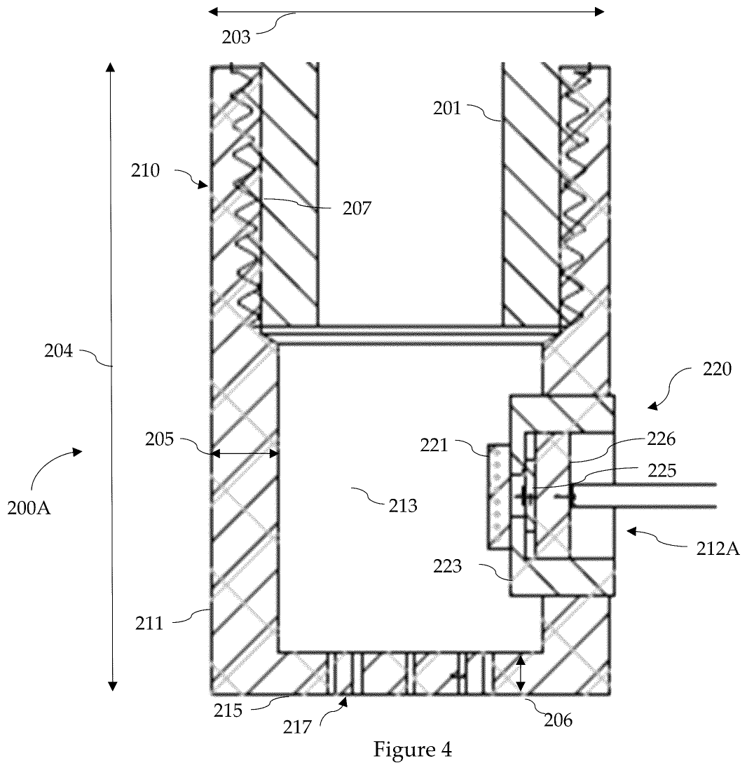

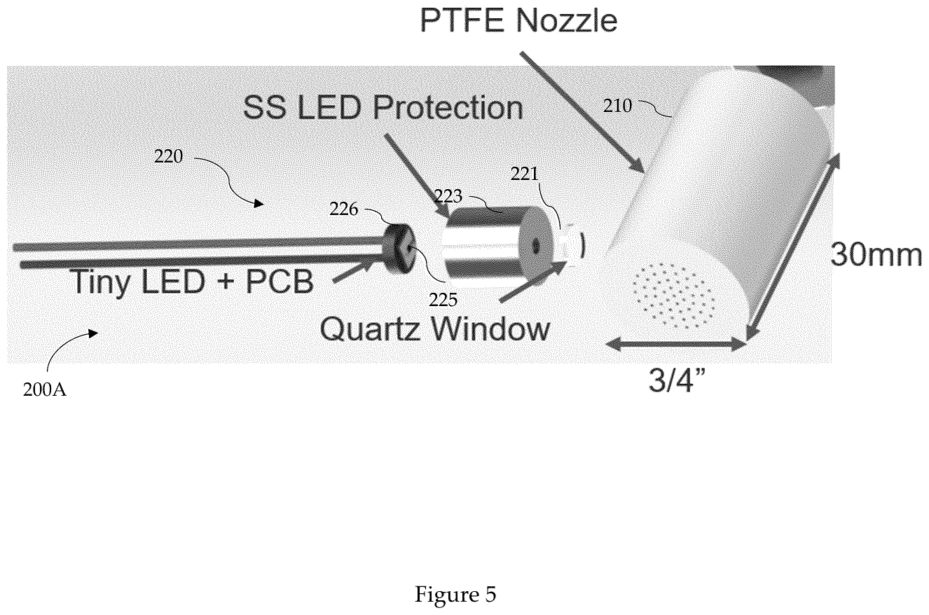

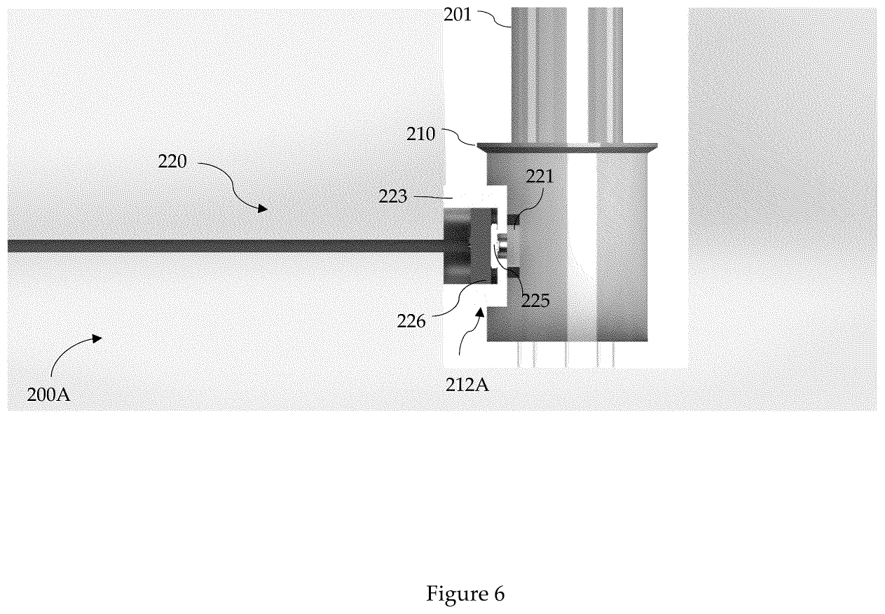

It should be appreciated that features of the nozzle 200 illustrated in FIGS. 2 and 3 may be present in other embodiments described herein, such as nozzle 200A in FIGS. 4-6.

FIGS. 4-6 show different views of another exemplary nozzle 200A. While the nozzle body 210 of nozzle 200 illustrated in FIGS. 2 and 3 included a thinned section 212 through which the light circuit 220 directed anti-microbial light into the inner chamber 213, the nozzle body 210 of the nozzle 200A has an aperture 212A in the side wall 211 through the lighting circuit 220 is inserted.

The light circuit 220 of nozzle 200A includes a housing 223 configured to absorb and disseminate heat produced by the lamp 225. The housing 223 may be comprised of a thermally conductive material and function as a heat sink for the lamp 225. Among other things, the housing 223 may be comprised of stainless steel or aluminum. The lamp 225 and PCB 226 may be inserted into the housing 223. In the illustrated embodiment, the PCB 226 and the housing 223 are cylindrical; however, the PCB 226 and the housing 223 may be other shapes.

The housing 223 may be coupled to the lamp 225 or PCB 226 by an adhesive, among other things. The adhesive may be thermally conductive such that heat generated by the lamp 225 is conducted by the adhesive to the housing 223. In some embodiments, the adhesive may be a thermally conductive epoxy. The adhesive may be used to join the front face of the lamp 225 to the housing 223. The housing 223 includes a housing aperture and the lamp 225 is positioned in alignment with the housing aperture so that anti-microbial light emitted from the lamp 225 is directed through the housing aperture and toward the inner chamber 213.

In order to protect the lamp 225 from the fluid flowing through the nozzle body 210, the light circuit 220 has a window 221 configured to be coupled to the housing 223 over the housing aperture. The window 221 may be comprised of a translucent material, such as quartz or cyclic block copolymers, among other things.

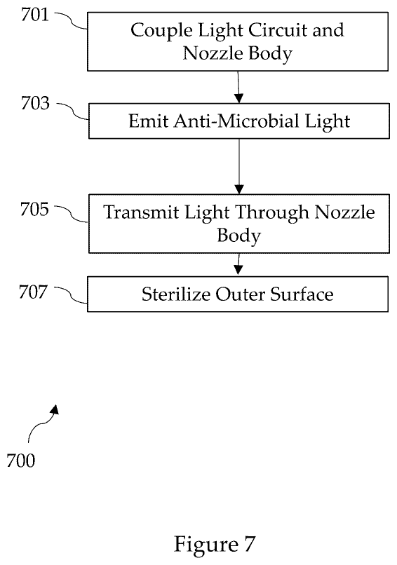

FIG. 7 shows a Process 700 for operating the nozzle 200 in accordance with various embodiments. It should be appreciated that a number of variations and modifications to Process 700 are contemplated including, for example, the omission of one or more aspects of Process 700, the addition of further conditionals and operations, or the reorganization or separation of operations and conditionals into separate processes. It should also be appreciated that while the Process 700 is primarily described with respect to the nozzle 200, the Process 700 may be used to operate other embodiments, such as the nozzle 200A.

The Process 700 begins by coupling the light circuit 220 to the nozzle body 210 in operation 701. In some embodiments, the light circuit 220 and the nozzle body 210 may be coupled by press fitting or gluing, among other things. Operation 701 may also include at least partially inserting the light circuit 220 into the side wall 211. Where the sidewall 211 has a thinned section 212, the light circuit 220 is positioned within the cavity formed by the thinned section 212. For example, the spacer 222 may be positioned between the thinned section 212 and the lamp 225. Where the side wall 211 has an aperture 212A, the light circuit 220 may be inserted into the aperture 212A.

The Process 700 proceeds to operation 703 where the lamp 225 emits the anti-microbial light toward the inner chamber of the nozzle body. For example, the lamp 225, such as a UVC LED, may emit the anti-microbial light in the direction of the thinned section 212 or the window 221. In some embodiments, the lamp 225 begins to emit the anti-microbial light in response to a trigger, such as liquid flowing through the nozzle, or a timer.

In operation 705, the anti-microbial light transmits through the nozzle body 210, including the side wall 211 and the tip 215, to the outer surface 214 of the nozzle body 210. Because the nozzle body 210 is comprised of semi-translucent material, a portion of the anti-microbial light is reflected within the inner chamber 213, while the remaining portion of the anti-microbial light is transmitted through the nozzle body 210 to the outer surface 214. The portions of light reflected and transmitted may be a function of, among other things, the nozzle body 210 material, the side wall thickness 205, and the tip thickness 206. Among other things, the nozzle body 210 may be configured to reflect 80-98% of the anti-microbial light within the inner chamber 213, while the remaining 2-20% transmits through the nozzle body 210.

The Process 700 proceeds to disinfecting the outer surface 214 of the nozzle body 210 in operation 707 after transmitting the portion of the anti-microbial light to the outer surface 214. The anti-microbial light disinfects the outer surface 214 by destroying or rendering harmless any microbial growth, or preventing microbial growth on the outer surface 214. To disinfect the outer surface 214 and inner chamber 213, the nozzle body 210 must be dosed with anti-microbial light of a sufficient intensity. The nozzle body 210 may be dosed for a time period until the intensity of the anti-microbial light is sufficient to prevent or destroy contamination. In some embodiments, the time period may be approximately 100 seconds, or until the light intensity for the outer surface of the nozzle body 210 reaches 3 mJ/cm² and the inner surface of the nozzle body 210 reaches 28 mJ/cm² , to give but a few examples. In some embodiments, the PCB 226 or another control circuit operates the lamp 225 to dose the nozzle body 210 in response to a standby time period. For example, the PCB 226 may dose the nozzle body 210 after a period of non-use of the fluid treatment system, such as a period of 3 hours or 1 day, among other time periods. In some embodiments, the PCB 226 operates the lamp 225 to dose the nozzle body 210 in response to a use trigger. For example, the control circuit may dose the nozzle body while fluid is flowing through the nozzle body 210. The PCB 226, or another control circuit, may also control the intensity of the anti-microbial light emitted from the lamp according to a safety threshold. For example, the lamp 225 emitting UVC anti-microbial light may only transmit an intensity of less than or equal to 3 mJ/cm² over an 8-hour period.

It is contemplated that the various aspects, features, processes, and operations from the various embodiments may be used in any of the other embodiments unless expressly stated to the contrary. Certain operations illustrated may be implemented by a computer executing a computer program product on a non-transient, computer-readable storage medium, where the computer program product includes instructions causing the computer to execute one or more of the operations, or to issue commands to other devices to execute one or more operations.

While the present disclosure has been illustrated and described in detail in the drawings and foregoing description, the same is to be considered as illustrative and not restrictive in character, it being understood that only certain exemplary embodiments have been shown and described, and that all changes and modifications that come within the spirit of the present disclosure are desired to be protected. It should be understood that while the use of words such as “preferable,” “preferably,” “preferred” or “more preferred” utilized in the description above indicate that the feature so described may be more desirable, it nonetheless may not be necessary, and embodiments lacking the same may be contemplated as within the scope of the present disclosure, the scope being defined by the claims that follow. In reading the claims, it is intended that when words such as “a,” “an,” “at least one,” or “at least one portion” are used there is no intention to limit the claim to only one item unless specifically stated to the contrary in the claim. The term “of” may connote an association with, or a connection to, another item, as well as a belonging to, or a connection with, the other item as informed by the context in which it is used. The terms “coupled to,” “coupled with” and the like include indirect connection and coupling, and further include but do not require a direct coupling or connection unless expressly indicated to the contrary. When the language “at least a portion” or “a portion” is used, the item can include a portion or the entire item unless specifically stated to the contrary. Unless stated explicitly to the contrary, the terms “or” and “and/or” in a list of two or more list items may connote an individual list item, or a combination of list items. Unless stated explicitly to the contrary, the transitional term “having” is open-ended terminology, bearing the same meaning as the transitional term “comprising.”

Various embodiments of the invention may be implemented at least in part in any conventional computer programming language. For example, some embodiments may be implemented in a procedural programming language (e.g., “C”), or in an object oriented programming language (e.g., “C++”). Other embodiments of the invention may be implemented as a pre-configured, stand-alone hardware element and/or as preprogrammed hardware elements (e.g., application specific integrated circuits, FPGAs, and digital signal processors), or other related components.

In an alternative embodiment, the disclosed apparatus and methods (e.g., see the various flow charts described above) may be implemented as a computer program product for use with a computer system. Such implementation may include a series of computer instructions fixed either on a tangible, non-transitory medium, such as a computer readable medium (e.g., a diskette, CD-ROM, ROM, or fixed disk). The series of computer instructions can embody all or part of the functionality previously described herein with respect to the system.

Those skilled in the art should appreciate that such computer instructions can be written in a number of programming languages for use with many computer architectures or operating systems. Furthermore, such instructions may be stored in any memory device, such as semiconductor, magnetic, optical or other memory devices, and may be transmitted using any communications technology, such as optical, infrared, microwave, or other transmission technologies.

Among other ways, such a computer program product may be distributed as a removable medium with accompanying printed or electronic documentation (e.g., shrink wrapped software), preloaded with a computer system (e.g., on system ROM or fixed disk), or distributed from a server or electronic bulletin board over the network (e.g., the Internet or World Wide Web). In fact, some embodiments may be implemented in a software-as-a-service model (“SAAS”) or cloud computing model. Of course, some embodiments of the invention may be implemented as a combination of both software (e.g., a computer program product) and hardware. Still other embodiments of the invention are implemented as entirely hardware, or entirely software.

The embodiments of the invention described above are intended to be merely exemplary; numerous variations and modifications will be apparent to those skilled in the art. Such variations and modifications are intended to be within the scope of the present invention as defined by any of the appended claims. It shall nevertheless be understood that no limitation of the scope of the present disclosure is hereby created, and that the present disclosure includes and protects such alterations, modifications, and further applications of the exemplary embodiments as would occur to one skilled in the art with the benefit of the present disclosure.

What is claimed is:

1. A liquid treatment system, comprising:
a nozzle body comprised of a semi-translucent material, the nozzle body including a side wall and a tip forming an inner chamber and an outer surface, wherein a side wall thickness and a tip thickness are a function of an anti-microbial light intensity range of the outer surface of the nozzle body; and
a light circuit configured to be coupled to the side wall and emit an anti-microbial light toward the inner chamber.

2. The liquid treatment system of claim 1, wherein the semi-translucent material includes a reflectivity in a range of 80-98%.

3. The liquid treatment system of claim 1, wherein the light circuit includes a light emitting diode configured to emit ultraviolet-C light.

4. The liquid treatment system of claim 1, wherein the anti-microbial light which has passed through the nozzle body to the outer surface of the nozzle body includes an intensity within the anti-microbial light intensity range, wherein the anti-microbial light is configured to disinfect the outer surface of the nozzle body while not exceeding 3 mJ/cm² at the outer surface over an 8-hour period.

5. The liquid treatment system of claim 4, wherein the tip includes a plurality of apertures.

6. The liquid treatment system of claim 4, wherein the light circuit is at least partially inserted into the side wall.

7. The liquid treatment system of claim 6, wherein the side wall includes a thinned section, and wherein the light circuit emits the anti-microbial light toward the inner chamber through the thinned section of the side wall.

8. The liquid treatment system of claim 7, wherein the light circuit includes an LED and a spacer, wherein the spacer is positioned between the thinned section of the side wall and the LED.

9. The liquid treatment system of claim 6, wherein the side wall includes an aperture, and the light circuit is configured to be inserted into the aperture.

10. The liquid treatment system of claim 9, wherein the light circuit includes
a translucent window; and
a housing coupled to the translucent window.

11. A method for operating a nozzle, comprising:
coupling a light circuit to a nozzle body including a side wall and a tip, the side wall and the tip forming an outer surface and an inner chamber;
emitting an anti-microbial light toward the inner chamber of the nozzle body;
transmitting a portion of the anti-microbial light to the outer surface of the nozzle body through the nozzle body; and
disinfecting the outer surface of the nozzle body after transmitting the portion of the anti-microbial light,
wherein the portion of the anti-microbial light includes an intensity not exceeding 3 mJ/cm² over an 8-hour period.

12. The method of claim 11, wherein the portion of the anti-microbial light is a function of a nozzle body material, a tip thickness, and a side wall thickness.

13. The method of claim 12, wherein the nozzle body material consists of a semi-translucent material with a reflectivity in a range of 80-98%.

14. The method of claim 11, wherein emitting the anti-microbial light occurs after determining a standby time period.

15. The method of claim 11, wherein the light circuit includes a light emitting diode configured to emit ultraviolet-C light.

16. The method of claim 11, wherein the tip includes a plurality of apertures.

17. The method of claim 11, wherein coupling the light circuit to the nozzle body includes at least partially inserting the light circuit into the side wall.

18. The method of claim 17, wherein the side wall includes a thinned section, and emitting the anti-microbial light toward the inner chamber of the nozzle body including emitting the anti-microbial light through the thinned section of the side wall.

19. The method of claim 18, wherein the light circuit includes an LED and a spacer, wherein coupling the light circuit to the nozzle body includes positioning the spacer between the thinned section of the side wall and the LED.

20. The method of claim 17, wherein the side wall includes an aperture, and coupling the light circuit to the nozzle body includes inserting the light circuit into the aperture.
